(19) United States
(12) Patent Application Publication
(10) Pub. No.: US 2024/0398953 A1
(43) Pub. Date: Dec. 5, 2024

(54) PSMA TARGETED ANTICANCER AGENT-PHTHALOCYANINE CONJUGATE COMPOUNDS AND USES THEREOF

(71) Applicant: CASE WESTERN RESERVE UNIVERSITY, Cleveland, OH (US)

(72) Inventors: James Basilion, Cleveland, OH (US); Xinning Wang, Cleveland, OH (US)

(21) Appl. No.: 18/672,853

(22) Filed: May 23, 2024

Related U.S. Application Data
(60) Provisional application No. 63/505,786, filed on Jun. 2, 2023.

(51) Int. Cl. A61K 41/00; A61K 31/695; A61P 35/00; C07F 7/02

(52) U.S. Cl. CPC A61K 41/0071; A61K 31/695; A61K 41/0042; A61P 35/00; C07F 7/025

(57) ABSTRACT

PSMA targeted anticancer agent-phthalocyanine conjugate compounds, pharmaceutical compositions comprising these compounds, methods for treating and detecting cancers in a subject.

### RELATED APPLICATION

This application claims priority from U.S. Provisional Application No. 63/505,786, filed Jun. 2, 2023. This application is also a Continuation-in-Part of U.S. application Ser. No. 18/708,899, filed May 9, 2024, which is a national stage entry of PCT/U2022/079566, filed Nov. 9, 2022, which claims priority to U.S. Patent Application No. 63/277,426, filed Nov. 9, 2021, 63/348,544, filed Jun. 3, 2022, and 63/359,257, filed Jul. 8, 2022, the subject matter of which are all incorporated herein by reference in their entirety.

### GOVERNMENT FUNDING

This invention was made with government support under CA204373 awarded by the National Institutes of Health; W81XWH-22-1-0784 awarded by the Department of Defense. The government has certain rights in the invention.

### BACKGROUND

Prostate-specific membrane antigen (PSMA) is a 120 kDa protein expressed in prostate tissues and was originally identified by reactivity with a monoclonal antibody designated 7E11-C5 (Horoszewicz et al., 1987, Anticancer Res. 7:927-935; U.S. Pat. No. 5,162,504). PSMA is characterized as a type II transmembrane protein sharing sequence identity with the transferrin receptor (Israeli et al., 1994, Cancer Res. 54:1807-1811). PSMA is a glutamate carboxy-peptidase that cleaves terminal carboxy glutamates from both the neuronal dipeptide N-acetylaspartylglutamate (NAAG) and gamma-linked folate polyglutamate. That is, expression of PSMA cDNA confers the activity of N-acetylated a-linked acidic dipeptidase or “NAALADase” activity (Carter et al., 1996, PNAS 93:749-753).

PSMA is expressed in increased amounts in prostate cancer, and elevated levels of PSMA are detectable in the sera of these patients (Horoszewicz et al., 1987, supra; Rochon et al., 1994, Prostate 25:219-223; Murphy et al., 1995, Prostate 26:164-168; and Murphy et al., 1995, Anticancer Res. 15:1473-1479). As a prostate carcinoma marker, PSMA is believed to serve as a target for imaging and cytotoxic treatment modalities for prostate cancer. Prostate carcinogenesis, for example, is associated with an elevation in PSMA abundance and enzymatic activity of PSMA. PSMA antibodies, particularly indium-111 labeled and tritium labeled PSMA antibodies, have been described and examined clinically for the diagnosis and treatment of prostate cancer. PSMA is expressed in prostatic ductal epithelium and is present in seminal plasma, prostatic fluid and urine.

Recent evidence suggests that PSMA is also expressed in tumor associated neovasculature of a wide spectrum of malignant neoplasms including conventional (clear cell) renal carcinoma, transitional cell carcinoma of the urinary bladder, testicular embryonal carcinoma, colonic adenocarcinoma, neuroendocrine carcinoma, gliobastoma multiforme, malignant melanoma, pancreatic ductal carcinoma, non-small cell lung carcinoma, soft tissue carcinoma, breast carcinoma, and prostatic adenocarcinoma. (Chang et al. (1999) Cancer Res. 59, 3192-3198).

In addition to prostate cancer and other proliferating or neoplastic cells, normal tissues can also express PSMA or PSMA-like molecules with the highest density of non-cancer tissue expression in the kidneys, lacrimal glands, and salivary glands. These tissues represent areas of interference (for PSMA-expressing cancer imaging) or dose-limiting sites of toxicity (for PSMA-targeted cancer therapies).

### SUMMARY

Embodiments described herein relate to PSMA targeted anticancer agent-phthalocyanine conjugate compounds, pharmaceutical compositions comprising these compounds, methods for treating and detecting cancers (e.g., prostate cancer) in a subject using these PSMA targeted anticancer agent-phthalocyanine conjugate compounds, and methods for identifying cancer cells (e.g., prostate cancer cells) in a sample using these compounds.

In some embodiments, the compound has the general formula (I):

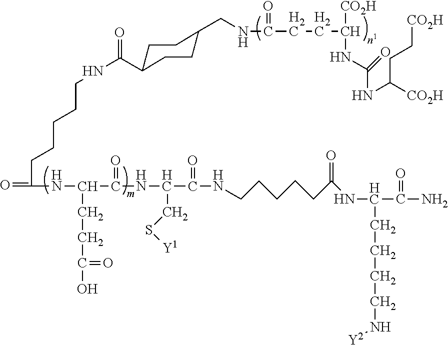

or a pharmaceutically acceptable salt thereof;
wherein m and n¹ are each independently 1, 2, 3, or 4;
Y¹ includes an anticancer agent that is directly or indirectly linked to the thiol group; and
Y² includes a phthalocyanine photosensitizer that is directly or indirectly linked to the amine group.

In some embodiments, the phthalocyanine photosensitizer having the formula:

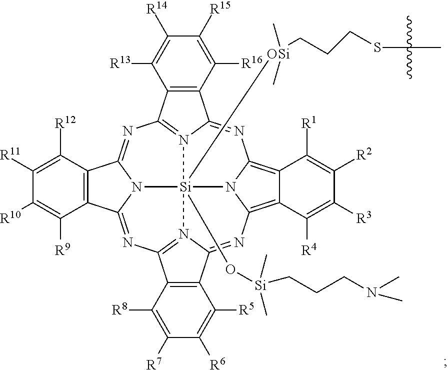

wherein R¹, R⁴, R⁵, R⁸, R⁹, R¹², R¹³, and R¹⁶ are each independently selected from the group consisting of hydrogen, halogen, nitro, cyano, hydroxyl, thiol, amino, and methyl; and
R², R³, R⁶, R⁷, R¹⁰, R¹¹, R¹⁴, and R¹⁵ are each independently selected from the group consisting of hydrogen, halogen, nitro, cyano, hydroxyl, thiol, amino, carboxy, aryl, heteroaryl, carbocyclyl, heterocyclyl, C₁₋₆ alkyl, C₁₋₆ alkenyl, C₁₋₆ alkynyl, C₁₋₆ alkoxy, C₁₋₆ acyl, C₁₋₆ alkylcarbonyloxy, C₁₋₆ carbocyclylalkyl, C₁₋₆ aminoalkyl, C₁₋₆ alkylamino, C₁₋₆ thioalkyl, C₁₋₆alkylthio, C₁₋₆hydroxyalkyl, C₁₋₆ alkyloxycarbonyl, C₁₋₆ alkylaminocarbonyl, and C₁₋₆ alkylcarbonylamino.

In some embodiments, R¹, R², R³, R⁴, R⁵, R⁶, R⁷, R⁸, R⁹, R¹⁰, R¹¹, R¹², R¹³, R¹⁴, R¹⁵ and R¹⁶ are each independently selected from the group consisting of hydrogen, halogen, nitro, cyano, hydroxyl, thiol, amino, and methyl.

In some embodiments, the phthalocyanine photosensitizer has the formula:

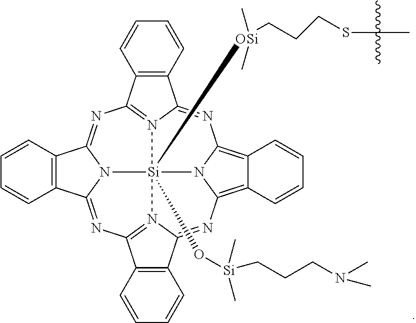

The phthalocyanine photosensitizer can be linked to the amine group via a non-cleavable linker, such as a succinimidyl 4-(N-maleimidomethyl)cyclohexane-1-carboxylate (SMCC) linker.

The anticancer agent can be linked to the thiol group via a protease cleavable or acid-labile linker. In some embodiments, the anticancer agent can be linked to the thiol group via a maleimido-caproyl linker-valine-citrulline cleavable peptide-p-aminobenzyl carbamate spacer (MC-VC-PABC) protease cleavable linker.

In some embodiments, Y¹ is a synthetic anticancer agent, such as monomethyl auristatin E (MMAE).

In particular embodiments, the compound can have the formula:

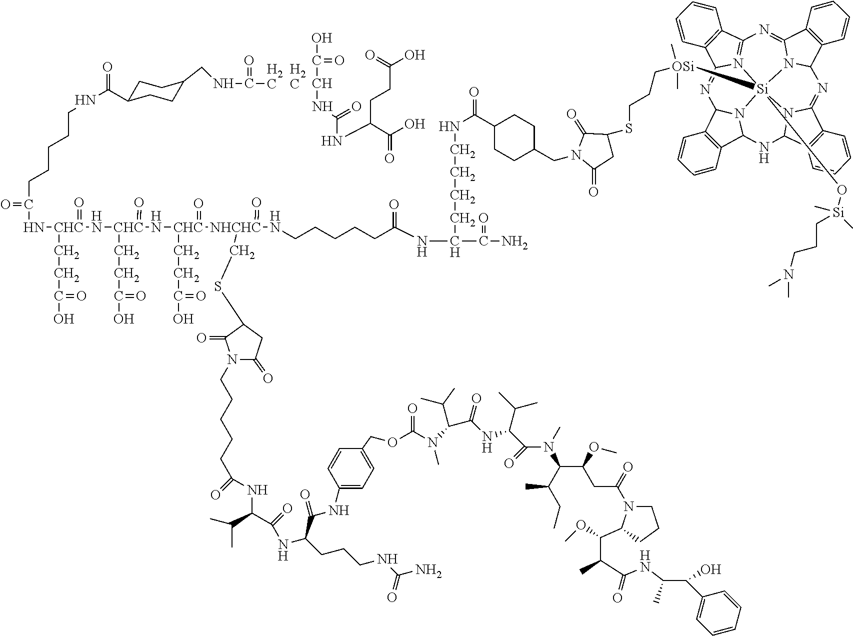

or a pharmaceutically acceptable salt thereof.

In certain embodiments, the compound can have a selectivity for PSMA expressing cancer tissue versus non-PSMA expressing non-cancer tissue≥5 times, ≥10 times, ≥20 times, ≥30 times, ≥40 times, ≥50 times or more times.

### BRIEF DESCRIPTION OF THE DRAWINGS

The following is a brief description of the drawings which are presented for the purpose of illustrating the invention and not for the purpose of limiting them.

FIG. 1 illustrates a synthesis scheme for Pc413.

FIG. 2 illustrates the structure of PSMA-1-MMAE-Pc413 where MMAE is linked to the thiol group of the PSMA-1 ligand portion of the compound via an MC-Val-Cit-PABC cleavable linker.

FIG. 3 illustrates Maestro Image data of mice receiving 100 nmol/kg of PSMA-1-MMAE-PC413 at different time points.

FIG. 4 illustrates Maestro Image data of mice receiving 100 nmol/kg of PSMA-1-MMAE-PC413 at different time points.

FIG. 5 illustrates a graph showing quantification of fluorescent signal intensity in PC3pip and PC3flu tumors. Values are mean±SD of 5 animals,

FIG. 6 illustrates ex vivo imaging of mouse organs at post-injection of 200 nmol-kg PSMA-1-MMAE-PC413. Fluorescent signal in PC3pip tumor was significantly higher than in other organs.

FIG. 7 illustrates ex vivo imaging of mouse organs at post-injection of 100 nmol-kg PSMA-1-MMAE-PC413. Fluorescent signal in PC3pip tumor and salivary gland was significantly higher than in other organs.

FIG. 8 illustrates ex vivo imaging of mouse organs at post-injection of 100 nmol-kg PSMA-1-MMAE-PC413. Fluorescent signal in PC3flu tumor, PC3pip tumor and salivary gland was significantly higher than in other organs.

FIG. 9 illustrates Maestro Image data of mice receiving 200 nmol/kg of PSMA-1-MMAE-PC413 at 24h post injection and 24 h post PDT.

FIGS. 10(A-B) illustrate in vivo antitumor activity of PSMA-1-MMAE-Pc413 in mice bearing heterotopic PC3pip tumors. For experiments, mice received drugs through tail vein injection. PDT was performed at 24-hour post injection. Treatment was scheduled every 7 days. Each group had 5 mice. For tumor growth curves and body weight curves, values are mean±SD of 5 animals. (A) Tumor growth curves of mice. (B) Body weight changes of mice treated with PSMA-1-MMAE-Pc413 with or without PDT.

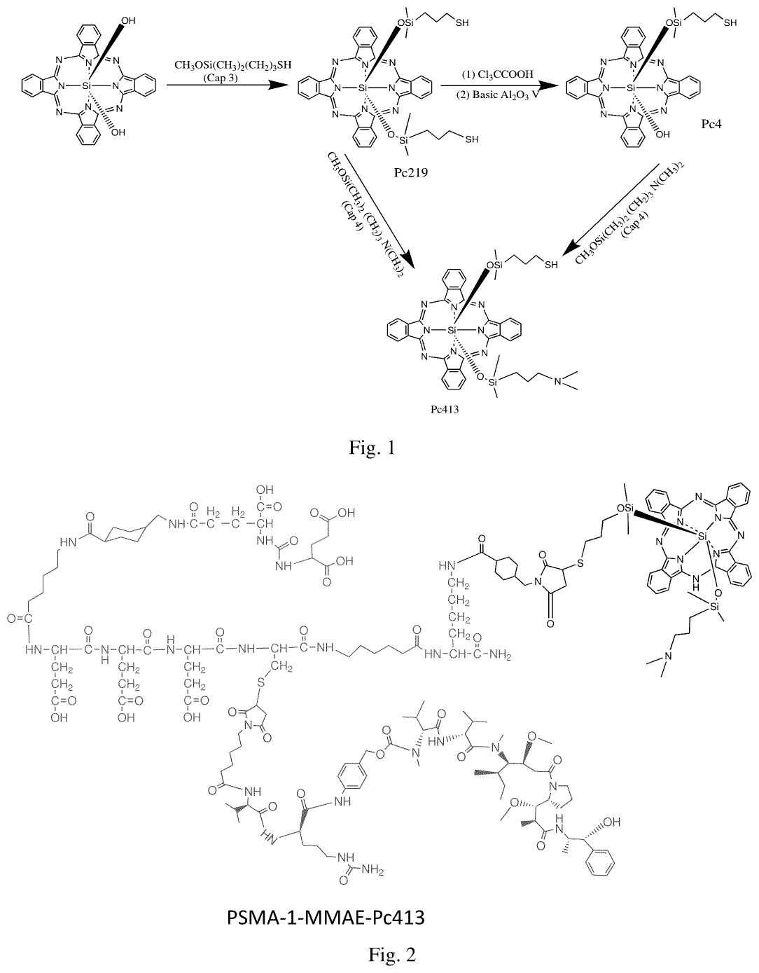

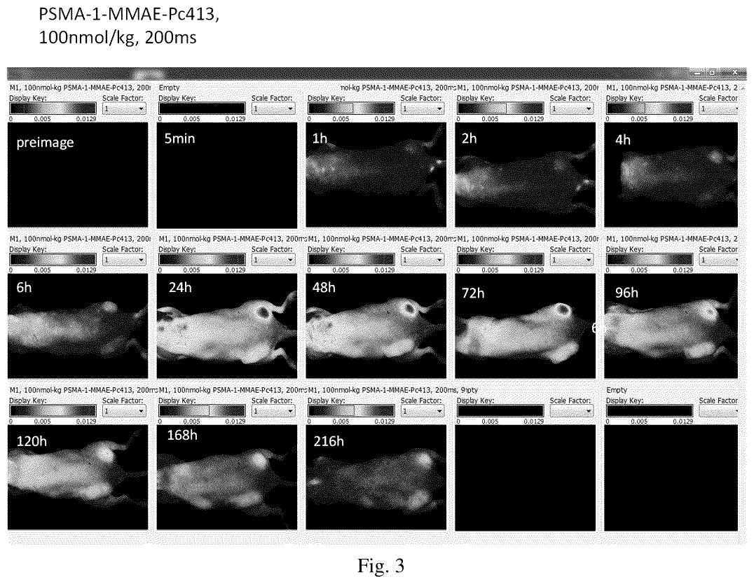

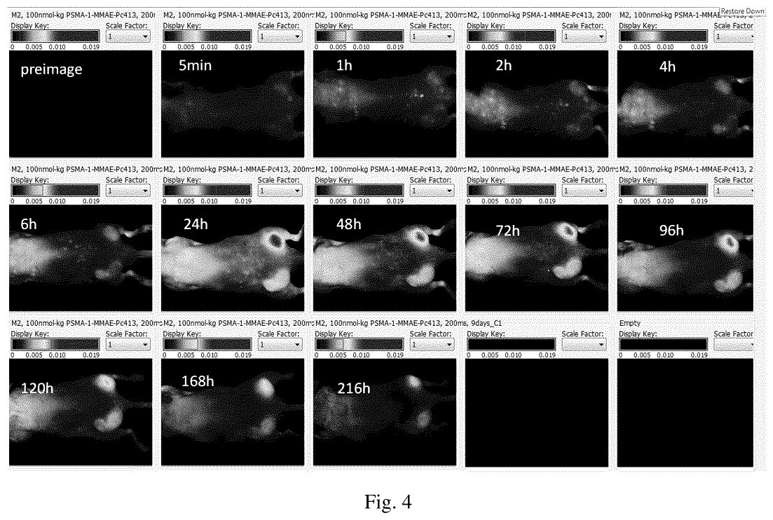

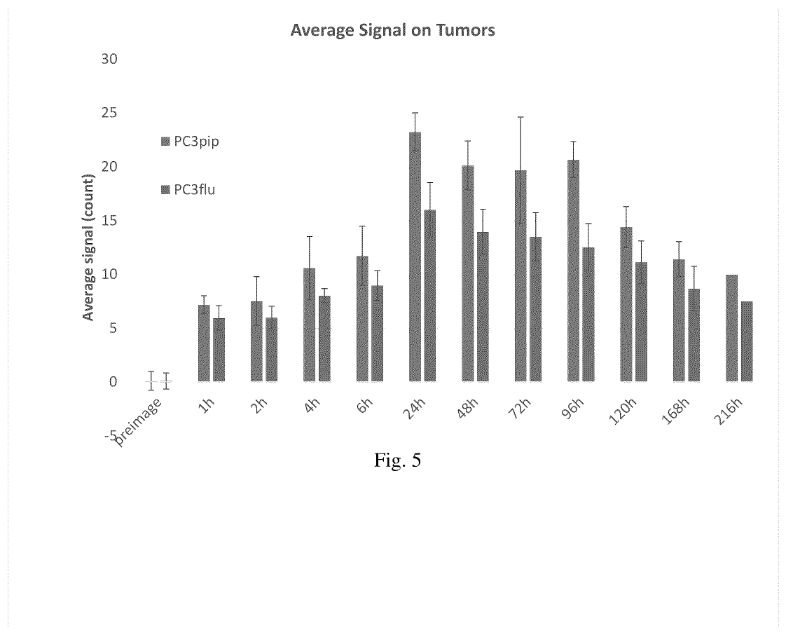

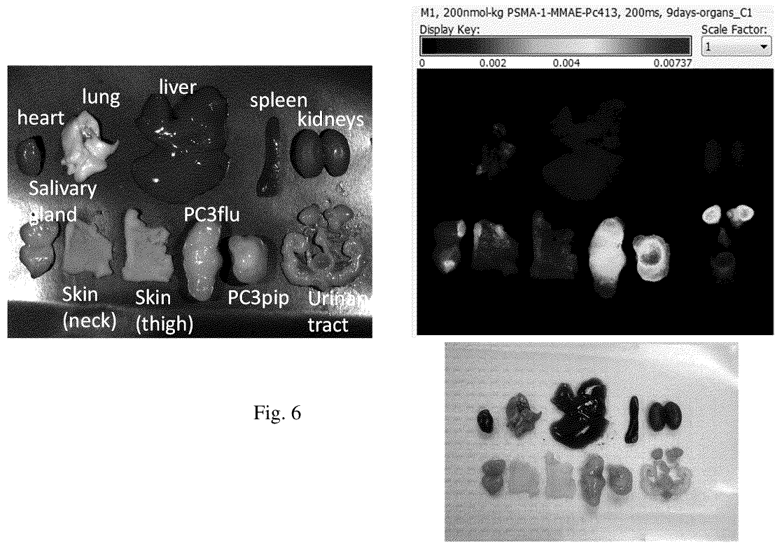

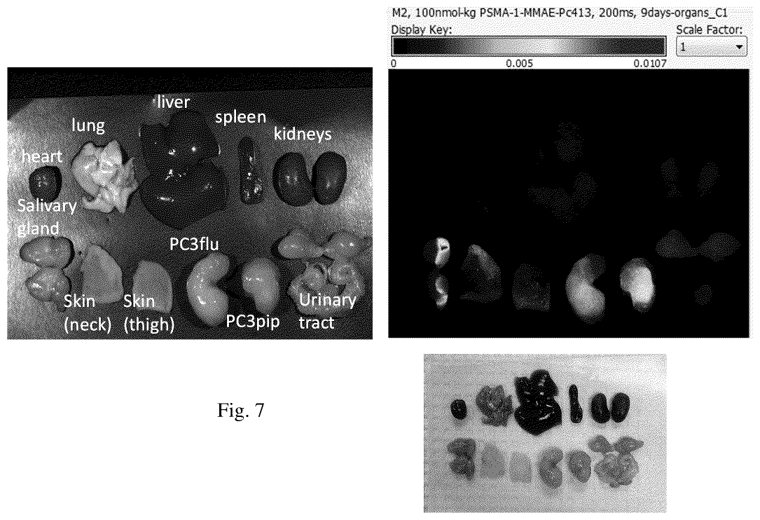

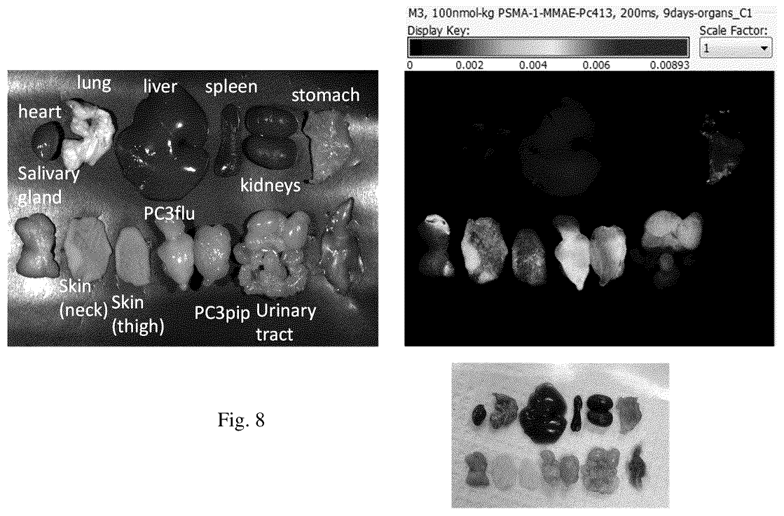

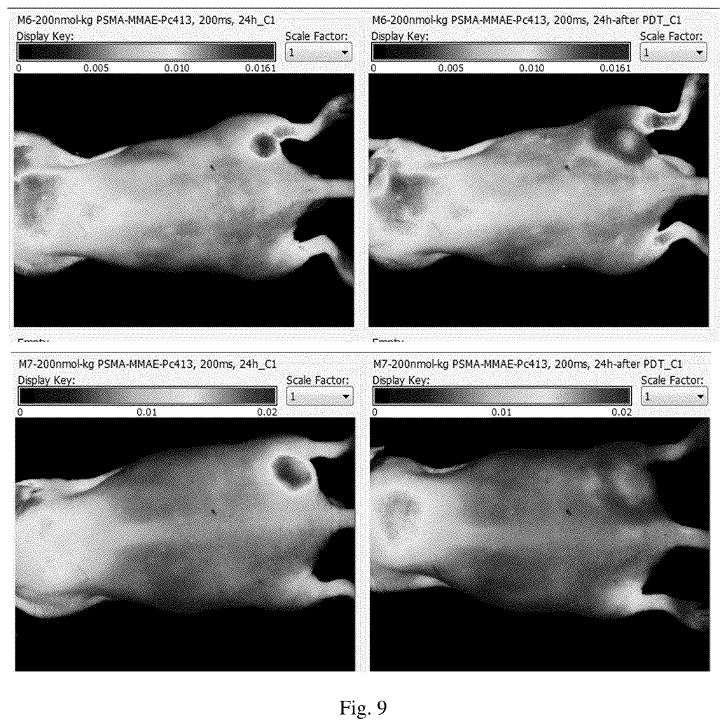

### DETAILED DESCRIPTION

All scientific and technical terms used in this application have meanings commonly used in the art unless otherwise specified. The definitions provided herein are to facilitate understanding of certain terms used frequently herein and are not meant to limit the scope of the application.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.,” as used herein are non-limiting and are for illustrative purposes only. “Including” and “including but not limited to” are used interchangeably.

The term “or” as used herein should be understood to mean “and/or” unless the context clearly indicates otherwise.

The term “agent” is used herein to denote a chemical compound, a mixture of chemical compounds, a biological macromolecule, or an extract made from biological materials.

The term “sample” can refer to a specimen or culture obtained from any source, as well as clinical, research, biological and environmental samples. Biological samples may be obtained from animals (including humans) and encompass cells, fluids, solids, tissues, and organs, and whole organisms.

The terms “patient”, “subject”, “mammalian host,” and the like are used interchangeably herein, and refer to humans and non-human animals (e.g., rodents, arthropods, insects, fish (e.g., zebrafish)), non-human primates, ovines, bovines, ruminants, lagomorphs, porcines, caprines, equines, or canines felines, aves, etc.).

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys, and liver.

Terms “cancer cell” or “tumor cell” can refer to cells that divide at an abnormal (i.e., increased) rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiform, meningoma, medulloblastoma, schwannoma and epidymoma.

The term “polypeptide” refers to a polymer composed of amino acid residues related naturally occurring structural variants, and synthetic non-naturally occurring analogs thereof linked via peptide bonds or modified peptide bonds (i.e., peptide isosteres), related naturally occurring structural variants, and synthetic non-naturally occurring analogs thereof, glycosylated polypeptides, and all “mimetic” and “peptidomimetic” polypeptide forms. Synthetic polypeptides can be synthesized, for example, using an automated polypeptide synthesizer. The term can refer to an oligopeptide, peptide, polypeptide, or protein sequence, or to a fragment, portion, or subunit of any of these. The term “protein” typically refers to large polypeptides. The term “peptide” typically refers to short polypeptides.

A “portion” of a polypeptide or protein means at least about three sequential amino acid residues of the polypeptide. It is understood that a portion of a polypeptide may include every amino acid residue of the polypeptide.

“Mutants,” “derivatives,” and “variants” of a polypeptide (or of the DNA encoding the same) are polypeptides which may be modified or altered in one or more amino acids (or in one or more nucleotides) such that the peptide (or the nucleic acid) is not identical to the wild-type sequence, but has homology to the wild type polypeptide (or the nucleic acid).

A “mutation” of a polypeptide (or of the DNA encoding the same) is a modification or alteration of one or more amino acids (or in one or more nucleotides) such that the peptide (or nucleic acid) is not identical to the sequences recited herein, but has homology to the wild type polypeptide (or the nucleic acid).

“Recombinant,” as used herein, means that a protein is derived from a prokaryotic or eukaryotic expression system.

The term “derivative” refers to an amino acid residue chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-benzylhistidine. Also included as derivatives are those amino acid residues, which contain one or more naturally occurring amino acid derivatives of the twenty standard amino acids, such as non-standard amino acids.

“PSMA” refers to Prostate Specific Membrane Antigen, a potential carcinoma marker that has been hypothesized to serve as a target for imaging and cytotoxic treatment modalities for cancer.

The term “small molecule” can refer to lipids, carbohydrates, polynucleotides,

polypeptides, or any other organic or inorganic molecules.

The term “imaging probe” can refer to a biological or chemical moiety that may be used to detect, image, and/or monitor the presence and/or progression of a cell cycle, cell function/physiology, condition, pathological disorder and/or disease.

The terms “treating” or “treatment” of a disease can refer to executing a treatment protocol to eradicate at least one diseased cell. Thus, “treating” or “treatment” does not require complete eradication of diseased cells.

The term “nanoparticle” refers to any particle having a diameter of less than 1000 nanometers (nm). In some embodiments, nanoparticles can be optically or magnetically detectable. In some embodiments, intrinsically fluorescent or luminescent nanoparticles, nanoparticles that comprise fluorescent or luminescent moieties, plasmon resonant nanoparticles, and magnetic nanoparticles are among the detectable nanoparticles that are used in various embodiments. In general, the nanoparticles should have dimensions small enough to allow their uptake by eukaryotic cells. Typically, the nanoparticles have a longest straight dimension (e.g., diameter) of 200 nm or less. In some embodiments, the nanoparticles have a diameter of 100 nm or less. Smaller nanoparticles, e.g., having diameters of 50 nm or less, e.g., about 1 nm to about 30 nm or about 1 nm to about 5 nm, are used in some embodiments.

An “effective amount” can refer to that amount of a therapeutic agent that results in amelioration of symptoms or a prolongation of survival in the subject and relieves, to some extent, one or more symptoms of the disease or returns to normal (either partially or completely) one or more physiological or biochemical parameters associated with or causative of the disease.

Therapeutic agents can include any agent (e.g., molecule, drug, pharmaceutical composition, etc.) capable of preventing, inhibiting, or arresting the symptoms and/or progression of a disease.

Embodiments described herein relate to PSMA targeted anticancer agent-phthalocyanine conjugate compounds, pharmaceutical compositions comprising these compounds, methods for treating and detecting cancers (e.g., prostate cancer) in a subject using these PSMA targeted anticancer agent-phthalocyanine conjugate compounds, and methods for identifying cancer cells (e.g., prostate cancer cells) in a sample using these compounds. It has been shown that PSMA targeted compounds conjugated to anticancer agents and phthalocyanine can increase uptake of the conjugate compound in PSMA expressing cells while also improving cell killing compared to agents administered alone. In addition, PSMA targeted anticancer agent-phthalocyanine conjugate compounds described herein can decrease non-PSMA target toxicity of the therapeutic agent, or a theranostic agent administered (e.g., systemically) to a subject.

Pathological studies indicate that PSMA is expressed by virtually all prostate cancers, and its expression is further increased in poorly differentiated, metastatic, and hormone-refractory carcinomas. Higher PSMA expression is also found in cancer cells from castration-resistant prostate cancer patients. Increased PSMA expression is reported to correlate with the risk of early prostate cancer recurrence after radical prostatectomy. In addition to being overexpressed in prostate cancer (PCa), PSMA is also expressed in the neovasculature of neoplasms including but not limited to conventional (clear cell) renal carcinoma, transitional cell carcinoma of the urinary bladder, testicular embryonal carcinoma, colonic adenocarcinoma, neuroendocrine carcinoma, gliobastoma multiforme, malignant melanoma, pancreatic ductal carcinoma, non-small cell lung carcinoma, soft tissue carcinoma, breast carcinoma, and prostatic adenocarcinoma.

In some embodiments, the PSMA targeted anticancer agent-phthalocyanine conjugate compounds described herein, can selectively recognize PSMA-expressing tumors, cancer cells, and/or cancer neovasculature in vivo and be used to deliver both an anticancer agent and a phthalocyanine photosensitizer to the PSMA-expressing tumors, cancer cells, and/or cancer neovasculature to treat and/or detect the PSMA-expressing tumors, cancer cells, and/or cancer neovasculature in a subject.

In some embodiments, the PSMA expressing cancer that is treated and/or detected is prostate cancer. In other embodiments, the cancer that is treated and/or detected can include malignant neoplasms, such a conventional (clear cell) renal carcinoma, transitional cell carcinoma of the urinary bladder, testicular embryonal carcinoma, colonic adenocarcinoma, neuroendocrine carcinoma, gliobastoma multiforme, malignant melanoma, pancreatic ductal carcinoma, non-small cell lung carcinoma, soft tissue carcinoma, breast carcinoma, and prostatic adenocarcinoma.

In some embodiments. the compound can include the general formula (I):

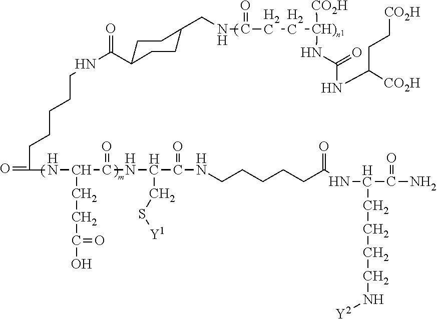

or a pharmaceutically acceptable salt thereof;
wherein m and n¹ are each independently 1, 2, 3, or 4;
Y¹ includes an anticancer agent that is directly or indirectly linked to the thiol group; and
Y² includes a phthalocyanine photosensitizer that is directly or indirectly linked to the amine group.

In some embodiments, the phthalocyanine photosensitizer has the formula:

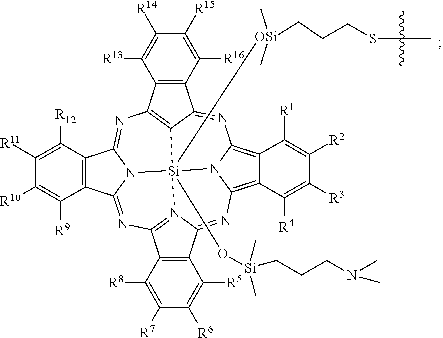

wherein R¹, R⁴, R⁵, R⁸, R⁹, R¹², R¹³, and R¹⁶ are each independently selected from the group consisting of hydrogen, halogen, nitro, cyano, hydroxyl, thiol, amino, and methyl; and
R², R³, R⁶, R⁷, R¹⁰, R¹¹, R¹⁴, and R¹⁵ are each independently selected from the group consisting of hydrogen, halogen, nitro, cyano, hydroxyl, thiol, amino, carboxy, aryl, heteroaryl, carbocyclyl, heterocyclyl, C₁₋₆ alkyl, C₁₋₆ alkenyl, C₁₋₆ alkynyl, C₁₋₆ alkoxy, C₁₋₆ acyl, C₁₋₆ alkylcarbonyloxy, C₁₋₆ carbocyclylalkyl, C₁₋₆ aminoalkyl, C₁₋₆ alkylamino, C₁₋₆ thioalkyl, C₁₋₆ alkylthio, C₁₋₆ hydroxyalkyl, C₁₋₆ alkyloxycarbonyl, C₁₋₆ alkylaminocarbonyl, and C₁₋₆ alkylcarbonylamino.

In some embodiments, R¹, R², R³, R⁴, R⁵, R⁶, R⁷, R⁸, R⁹, R¹⁰, R¹¹, R¹², R¹³, R¹⁴, R¹⁵ and R¹⁶ are each independently selected from the group consisting of hydrogen, halogen, nitro, cyano, hydroxyl, thiol, amino, and methyl.

In other embodiments, the phthalocyanine photosensitizer can have the formula:

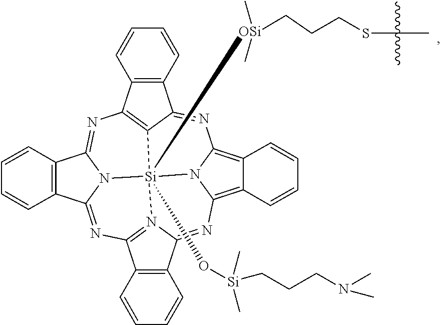

wherein R¹, R⁴, R⁵, R⁸, R⁹, R¹², R¹³, and R¹⁶ are each independently selected from the group consisting of hydrogen, halogen, nitro, cyano, hydroxyl, thiol, amino, and methyl; and
R², R³, R⁶, R⁷, R¹⁰, R¹¹, R¹⁴, and R¹⁵ are each independently selected from the group consisting of hydrogen, halogen, nitro, cyano, hydroxyl, thiol, amino, carboxy, aryl, heteroaryl, carbocyclyl, heterocyclyl, C₁₋₆ alkyl, C₁₋₆ alkenyl, C₁₋₆ alkynyl, C₁₋₆ alkoxy, C₁₋₆ acyl, C₁₋₆ alkylcarbonyloxy, C₁₋₆ carbocyclylalkyl, C₁₋₆ aminoalkyl, C₁₋₆ alkylamino, C₁₋₆ thioalkyl, C₁₋₆ alkylthio, C₁₋₆ hydroxyalkyl, C₁₋₆ alkyloxycarbonyl, C₁₋₆ alkylaminocarbonyl, and C₁₋₆ alkylcarbonylamino.

In some embodiments, R¹, R², R³, R⁴, R⁵, R⁶, R⁷, R⁸, R⁹, R¹⁰, R¹¹, R¹², R¹³, R¹⁴, R¹⁵ and R¹⁶ are each independently selected from the group consisting of hydrogen, halogen, nitro, cyano, hydroxyl, thiol, amino, and methyl.

In still other embodiments, the phthalocyanine photosensitizer can have the formula:

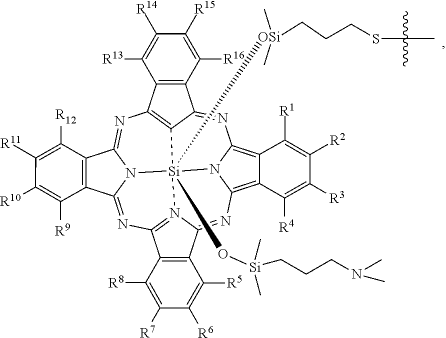

wherein R¹, R⁴, R⁵, R⁸, R⁹, R¹², R¹³, and R¹⁶ are each independently selected from the group consisting of hydrogen, halogen, nitro, cyano, hydroxyl, thiol, amino, and methyl; and
R², R³, R⁶, R⁷, R¹⁰, R¹¹, R¹⁴, and R¹⁵ are each independently selected from the group consisting of hydrogen, halogen, nitro, cyano, hydroxyl, thiol, amino, carboxy, aryl, heteroaryl, carbocyclyl, heterocyclyl, C₁₋₆ alkyl, C₁₋₆ alkenyl, C₁₋₆ alkynyl, C₁₋₆ alkoxy, C₁₋₆ acyl, C₁₋₆ alkylcarbonyloxy, C₁₋₆ carbocyclylalkyl, C₁₋₆ aminoalkyl, C₁₋₆ alkylamino, C₁₋₆ thioalkyl, C₁₋₆alkylthio, C₁₋₆hydroxyalkyl, C₁₋₆ alkyloxycarbonyl, C₁₋₆ alkylaminocarbonyl, and C₁₋₆ alkylcarbonylamino.

In some embodiments, R¹-R¹⁶ are independently selected from the group consisting of hydrogen, halogen, nitro, cyano, hydroxyl, thiol, amino, and methyl.

In some embodiments, the Y² can include the phthalocyanine photosensitizer having the formula:

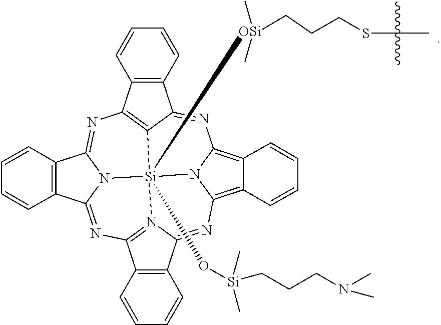

A PSMA targeted anticancer-phthalocyanine conjugate compound can include a phthalocyanine photosensitizer directly or indirectly coupled to the amine group of the PSMA targeted portion of the compound. The phthalocyanine photosensitizer can be coupled directly or indirectly to the PSMA targeted portion of the compound via a linker. In some embodiments, the phthalocyanine photosensitizer is linked to the amine group of the PSMA targeted compound having formula (I) via a non-cleavable linker, such as a succinimidyl 4-(N-maleimidomethyl)cyclohexane-1-carboxylate (SMCC) linker.

In some embodiments, a PSMA targeted anticancer-phthalocyanine conjugate compound can have the following general formula:

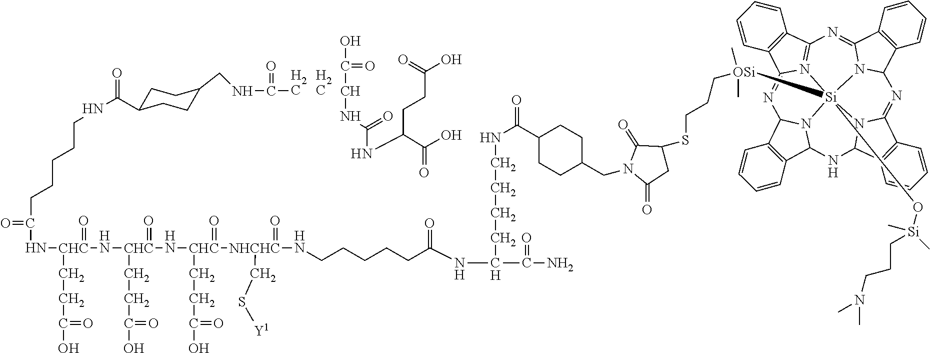

or a pharmaceutically acceptable salt thereof,
wherein Y¹ is an anti-cancer agent or chemotherapeutic, that is directly or indirectly linked to the thiol group.

This PSMA targeted anticancer-Pc413 conjugate compound can be prepared by linking the cysteine residue of the PSMA ligand to a lysine residue via a C6 linker, which then allows for coupling the phthalocyanine photosensitizer, Pc413 to the amine on the lysine amino acid.

In some embodiments, the anticancer agent can be coupled directly or indirectly to the PSMA targeted portion of the compound via a linker. The linker can include a heterobifunctional crosslinker capable of conjugating to the cysteine residue of the PSMA targeted portion of the compound via a maleimide group that is sulfhydryl (thiol; —SH) reactive.

In other embodiments, the heterobifunctional crosslinker can include a protease cleavable or acid-labile linker. In particular embodiments, the anticancer agent can be coupled directly or indirectly to the PSMA targeted compound via an acid-labile linker, such as a 4-(4-maleimidomethyl)cyclohexane-1-carboxyl hydrazide (MMCCH) linker. The MMCCH linker includes a hydrazine bond that can be cleaved to release the anticancer agent coupled to the PSMA targeted compound.

In particular embodiments, the anticancer agent can be coupled directly or indirectly to the PSMA targeted portion of the compound (PSMA-1(Cys)) via a protease cleavable linker. The protease cleavable linker can include a MC-Val-Cit-PABC protease cleavable linker where the Valine-Citrulline-PABC portion is protease cleavable.

In some embodiments, the anticancer agent includes the antimitotic agent, monomethyl auristatin E (MMAE). In some embodiments, the PSMA targeted anticancer-phthalocyanine conjugate compounds described herein are prepared by coupling a “vc-MMAE” linker-drug combination construct to the thiol group of the cysteine residue of the PSMA targeted portion of the compound. The vc-MMAE construct is also referred to as the linker-drug mc-vc-PABC-MMAE. The construct utilizes a maleimidocaproyl (mc) spacer, a protease-sensitive dipeptide, valine-citrulline (vc), a self-immolative spacer, para-amino benzyloxycarbonyl (PABC), and the antimitotic agent, MMAE. The mc spacer provides enough room for the vc group to be recognized by the lysosomal cysteine protease cathepsin B in a PSMA expressing cell, which cleaves the citrulline-PABC amide bond. The resultant PABC-substituted MMAE is not a stable intermediate and spontaneously undergoes a 1,6-elimination with a loss of p-iminoquinone methide and carbon dioxide (self-immolation) leaving MMAE as the product to exert anti-mitotic effects.

In some embodiments, PSMA targeted anticancer-phthalocyanine conjugate compounds provide targeted delivery of the anticancer agent, while also allowing for tumor imaging and targeted photodynamic therapy.

In an embodiment, a PSMA targeted anticancer-phthalocyanine conjugate compound coupled to the anticancer agent MMAE via a MC-VC-PABC protease cleavable linker and coupled to the near-infrared labeling agent or photosensitizer Pc413 (i.e., a PSMA-1-VcMMAE-Pc413 conjugate compound) can have the formula:

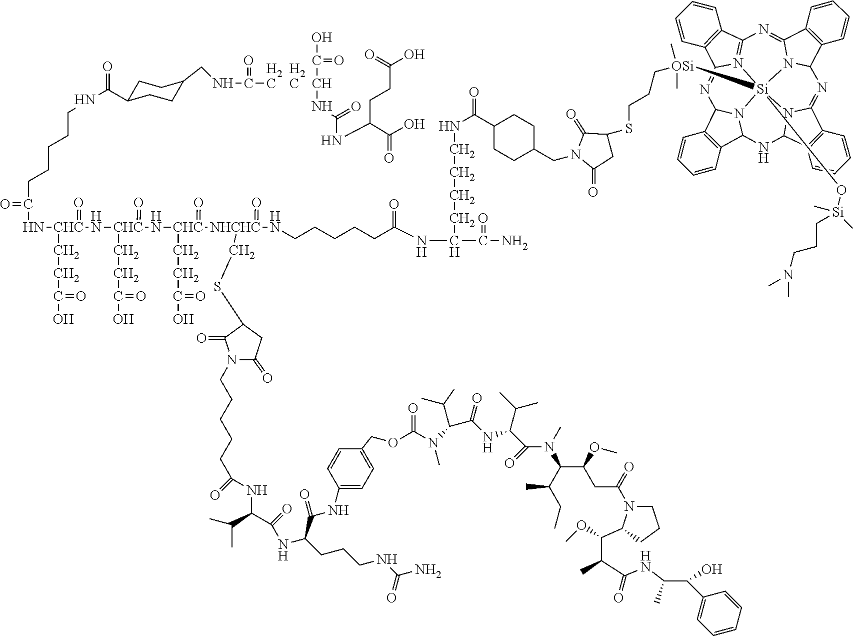

or a pharmaceutically acceptable salt thereof.

In some aspects, the PSMA targeted anticancer-phthalocyanine conjugate compound described herein may be used in conjunction with non-invasive imaging techniques for in vivo imaging to determine the location or distribution of cancer cells. The term “in vivo imaging” refers to any method, which permits the detection of a labeled PSMA targeted conjugate compound, as described above.

The PSMA targeted anticancer-phthalocyanine conjugate compound described herein can be administered to the subject by, for example, systemic, topical, and/or parenteral methods of administration. These methods include, e.g., injection, infusion, deposition, implantation, or topical administration, or any other method of administration where access to the tissue is desired. In one example, administration can be by intravenous injection. Single or multiple administrations of the compound can be given.

In some embodiments, the PSMA targeted anticancer-phthalocyanine conjugate compound described herein can be administered to a subject in a detectable quantity. The detectable quantity can be an amount of the compound administered that is sufficient to enable detection of binding of the compound to the cancer cells. The detectable quantity can be an imaging effective quantity that is sufficient to enable imaging of binding of the compound to the cancer cells.

The PSMA targeted anticancer-phthalocyanine conjugate compound described herein administered to a subject can be used to determine the presence, location, and/or distribution of cancer cells, i.e., PSMA expressing cancer cells or PSMA expressing neovaculature of the cancer cells, in an organ or body area of a patient. The presence, location, and/or distribution of the PSMA ligands coupled to a detectable moiety in the animal's tissue, e.g., brain tissue, can be visualized (e.g., with an in vivo imaging modality). “Distribution” as used herein is the spatial property of being scattered about over an area or volume. In this case, “the distribution of cancer cells” is the spatial property of cancer cells being scattered about over an area or volume included in the animal's tissue, e.g., prostate tissue. The distribution of the PSMA targeted anticancer-phthalocyanine conjugate compound may then be correlated with the presence or absence of cancer cells in the tissue. A distribution may be dispositive for the presence or absence of a cancer cells or may be combined with other factors and symptoms by one skilled in the art to positively detect the presence or absence of migrating or dispersing cancer cells, cancer metastases or define a tumor margin in the subject.

The PSMA targeted anticancer-phthalocyanine conjugate compound can be used in intra-operative imaging techniques to guide surgical resection and eliminate the “educated guess” of the location of the tumor by the surgeon. Previous studies have determined that more extensive surgical resection improves patient survival. Thus, PSMA targeted conjugate compounds that function as diagnostic molecular imaging agents have the potential to increase patient survival rates. PSMA-targeted conjugates are easily detectable at real-time imaging exposures (i.e., at <67 ms) and thus capable of being used for real-time image-guided surgery (IGS) during urological surgery. Thus, the compositions described herein for use in IGS can increase patient survival rates especially when including a therapeutic agent and/or when combined with subsequent photodynamic therapy (PDT) in accordance with a method described herein.

In some embodiments, IGS can be performed real-time using an in vivo imaging system, such as an intraoperative near-infrared fluorescence imaging system. In certain embodiments, image guided surgery can be performed using a FLARE (Fluorescence-Assisted Resection and Exploration) intraoperative NIR fluorescence imaging system where the targeted cancer is imaged at a wavelength of about 665 nm to about 705 nm. In an exemplary embodiment, the targeted PMSA expressing cancer is imaged using a PSMA targeted anticancer-phthalocyanine conjugate compound during image guided surgical resection at about 668 to about 690 nm.

Following administration, detection/localization of PSMA-targeted conjugate compounds, and surgical resection of the targeted cancer cells, remaining or residual PSMA expressing cancer cells that have penetrated beyond the resection site may revert to a proliferative state to produce a more aggressive recurrent tumor that continues to disperse into nonneoplastic tissue adjacent the resection site and beyond. The PSMA targeted anticancer-phthalocyanine conjugate compounds that are bound to and/or complexed with the PSMA expressing cancer cells remaining in, and/or adjacent to, the surgical site can then be irradiated to induce the cytotoxic effects of the coupled phthalocyanine, such as Pc413, on residual cancer cells.

Methods and photosensitizing agents for conducting photodynamic therapy (PDT) or photothermal therapy (PTT) are known in the art. See for example Thierry Patrice. Photodynamic Therapy; Royal Society of Chemistry, 2004. PDT is a site-specific treatment modality that requires the presence of a photosensitizer, light, and adequate amounts of molecular oxygen to destroy targeted tumors (Grossweiner, Li, The science of phototherapy. Springer: The Netherlands, 2005). Upon illumination, a photoactivated sensitizer transfers energy to molecular oxygen that leads to the generation of singlet oxygen (O²) and other reactive oxygen species (ROS), which initiate apoptosis and oxidative damage to cancer cells. Only the cells that are exposed simultaneously to the targeted PDT compound (which is non-toxic in the dark) and light are destroyed while surrounding healthy, non-targeted and nonirradiated cells are spared from photodamage. Furthermore, the fluorescence of the phthalocyanine photosensitizing compound, such as Pc413, coupled to the PSMA targeting moiety enable simultaneous diagnostic optical imaging that can be used to guide the PDT step of the method of treating cancer described herein.

The PSMA targeted anticancer-phthalocyanine conjugate compound described herein can include phthalocyanine photosensitizers that are excited by an appropriate light source to produce radicals and/or reactive oxygen species. Typically, when a sufficient amount of photosensitizer, such as a phthalocyanine photosensitizer, appears in diseased tissue (e.g., tumor tissue), the photosensitizer can be activated by exposure to light for a specified period. The light dose supplies sufficient energy to stimulate the photosensitizer, but not enough to damage neighboring healthy tissue. The radicals or reactive oxygen produced following photosensitizer excitation kill the target cells (e.g., cancer cells). In some embodiments, the targeted tissue can be locally illuminated. The light, which is capable of activating the photosensitizer can delivered to the targeted cancer cells, using for example, semiconductor laser, dye laser, optical parametric oscillator or the like. For example, light can be delivered to a photosensitizer via an argon or copper pumped dye laser coupled to an optical fiber, a double laser consisting of KTP (potassium titanyl phosphate)/YAG (yttrium aluminum garnet) medium, LED (light emitting diode), or a solid state laser. It will be appreciated that any source light can be used as long as the light excites the phthalocyanine photosensitizer bound or complexed with a PSMA expressing cancer cell.

In some embodiments, the surgical resection site can be irradiated using visible laser diodes to photoactivate the phthalocyanine photosensitizer coupled to the PSMA targeting moiety portion of the compound. For example, in some embodiments, where the phthalocyanine photosensitizer coupled to the PSMA targeting moiety is Pc413, the surgical resection site can be irradiated using visible laser diodes emitting at 668 nm. In certain embodiments, the surgical resection site can be irradiated with an amount of radiation effective to inhibit tumor recurrence in the subject. In an exemplary embodiment, the PDT step of the method of treating cancer described herein can include irradiating the resection site bed with a peak wavelength of 668 nm at 300 mW power for a total radiant exposure of 150 J/cm2. It is contemplated that the absorption peak of Pc413 at near infra-red permits increased light penetration across tissues. Photoactivation of Pc413 specifically kills PSMA-expressing tumor cells while sparing the tumor microenvironment as well as the surrounding healthy tissues.

Following PDT, the resection site of a subject can be further imaged and irradiated after a period of time(s) to detect and ablate residual PSMA expressing cancer cells that may have survived previous irradiation. This optional step may or may not include additional administration of a pharmaceutical composition including a PSMA targeted anticancer-phthalocyanine conjugate compound described herein.

The PSMA targeted anticancer-phthalocyanine conjugate compound can also be used to deliver a therapeutic anticancer agent to a PSMA expressing cancer cell of a subject or mammal. Therefore, some embodiments relate to the administration of a PSMA targeted anticancer-phthalocyanine conjugate compound to a subject having or suspected of having cancer, such as a PSMA expressing cancer for the treatment of cancer. In particular embodiments, a PSMA targeted anticancer-phthalocyanine conjugate compound can be administered to a subject having prostate cancer.

The PSMA targeted anticancer-phthalocyanine conjugate compound can target and transiently interact with, bind to, and/or couple with a cancer cell, such as a prostate cancer cell, and once interacting with, bound to, or coupled to the targeted cell or tissue advantageously facilitate delivery of the anticancer agent within cell by, for example, receptor mediated endocytosis. In certain embodiments, the anticancer agent coupled to a PSMA targeted portion of the conjugate compound is an antimitotic agent such as ve monomethyl auristatin E (vcMMAE). Without being bound by theory, it is believed that once a PSMA targeted MMAE-phthalocyanine conjugate compound is delivered to a PSMA expressing cell, receptor-mediated internalization of the compound leads to lysosomal protease release of the anti-mitotic agent, allowing MMAE to inhibit cell division by blocking the polymerization of tubulin.

Examples of additional anticancer agents that can be directly or indirectly coupled to a PSMA targeted anticancer-phthalocyanine conjugate compound as described herein can include, but are not limited to chemotherapeutic agents and other agents including Taxol, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; ctanidazole; ctoposide; ctoposide phosphate; etoprine; fadrozole hydrochloride; fazarabine; fenretinide; floxuridine; fludarabine phosphate; fluorouracil; fluorocitabine; fosquidone; fostriccin sodium; gemcitabine; gemcitabine hydrochloride; hydroxyurca; idarubicin hydrochloride; ifosfamide; ilmofosine; interleukin II (including recombinant interleukin II, or rIL2), interferon α-2a; interferon α-2b; interferon α-n1; interferon α-n3; interferon β-I a; interferon γ-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other examples of anti-cancer agents include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antincoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; silicon phthalocyanine (PC4) sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans (GAGs); tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Still other examples of anti-cancer agents can include the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Arnad), AM-138 (Armad/Kyowa Hakko), IDN-5005 (Indena), Cryptophycin 52 (also known as LY-355703), AC-7739 (Ajinomoto, also known as AVE-8063A and CS-39.HCI), AC-7700 (Ajinomoto, also known as AVE-8062, AVE-8062A, CS-39-L-Scr.HCI, and RPR-258062A), Vitilevuamide, Tubulysin A, Canadensol, Centaurcidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin Al (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapinc, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-IAABE (Cytoskeleton/Mt. Sinai School of Medicine), A-204197 (Abbott), T-607 (Tularik, also known as T-900607), RPR-115781 (Aventis), Elcutherobins (such as Desmethyleleutherobin, Desactyleleutherobin, Isocleutherobin A, and Z-Elcutherobin), Caribacoside, Caribacolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

Other examples of anti-cancer agents include alkylating agents, such as nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosourcas (e.g., carmustine, lomusitne, semustine, streptozocin, etc.), or triazenes (decarbazine, etc.), antimetabolites, such as folic acid analog (e.g., methotrexate), or pyrimidine analogs (e.g., fluorouracil, floxouridine, Cytarabine), purine analogs (e.g., mercaptopurine, thioguanine, pentostatin, vinca alkaloids (e.g., vinblastin, vincristine), epipodophyllotoxins (e.g., etoposide, teniposide), platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, amino glutethimide).

In an embodiment, PSMA-1-VcMMAE-Pc413 conjugate compounds administered to a subject can provide for targeted delivery of the MMAE chemotherapeutic agent to PSMA+prostate cancer cells while the Pc413 coupled to PSMA-1 allows for image guidance during prostate tumor resection and subsequent targeted PDT to eliminate unresectable or remaining cancer cells. In an exemplary embodiment, a pharmaceutical composition including the PSMA-1-VcMMAE-Pc413 conjugate compound is administered to a subject about every 7 days and PDT is performed on the subject 24 hours post injection.

The PSMA targeted anticancer-phthalocyanine conjugate compound can be administered alone as a monotherapy, or in conjunction with or in combination with one or more additional therapeutic agents. In some embodiments, a PSMA targeted anticancer-phthalocyanine conjugate compound described herein can be administered to the subject in combination with an additional anti-cancer agent. In a particular embodiment, a PSMA targeting conjugate compound coupled to vcMMAE as described herein can be administered to the subject in combination with a MAPK inhibitor.

It will be appreciated that additional detectable moieties, therapeutic agents, and/or theranostic agents administered to a subject need not be conjugated directly or indirectly to the PSMA targeting portion of the PSMA targeted anticancer-phthalocyanine conjugate compound and can optionally be provided in a pharmaceutical composition or preparation with the PSMA targeted anticancer-phthalocyanine conjugate compounds described herein or in a separate pharmaceutical composition.

The term “in conjunction with” or “in combination with” indicates that the PSMA targeted anticancer-phthalocyanine conjugate compound is administered at about the same time as the additional agent. The PSMA targeted anticancer-phthalocyanine conjugate compound can be administered to the subject in need thereof as part of a pharmaceutical composition comprising the compound and a pharmaceutically acceptable carrier or excipient and, optionally, one or more additional therapeutic agents. The compound and additional therapeutic agent can be components of separate pharmaceutical compositions, which can be mixed together prior to administration or administered separately. The PSMA targeted anticancer-phthalocyanine conjugate compound can, for example, be administered in a composition containing the additional therapeutic agent, and thereby, administered contemporaneously with the agent. Alternatively, the PSMA targeted anticancer-phthalocyanine conjugate compound can be administered contemporancously, without mixing (e.g., by delivery of the compound on the intravenous line by which the compound is also administered, or vice versa). In another embodiment, the PSMA targeted anticancer-phthalocyanine conjugate compound can be administered separately (e.g., not admixed), but within a short timeframe (e.g., within 24 hours) of administration of the compound.

The disclosed PSMA targeted anticancer-phthalocyanine conjugate compound and additional therapeutic agents, detectable moieties, and/or theranostic agents described herein can be administered to a subject by any conventional method of drug administration. Delivery can be by injection into the brain or body cavity of a patient. Delivery can be in vivo, or ex vivo. Administration can be local or systemic as indicated. More than one route can be used concurrently, if desired. The preferred mode of administration can vary depending upon the particular disclosed compound chosen. In specific embodiments, parenteral or systemic administration (e.g., intravenous) are preferred modes of administration for treatment.

The methods described herein contemplate either single or multiple administrations, given either simultaneously or over an extended period of time. A PSMA targeted anticancer-phthalocyanine conjugate compound (or pharmaceutical composition containing the compound) can be administered at regular intervals, depending on the nature and extent of the inflammatory disorder's effects, and on an ongoing basis. Administration at a “regular interval,” as used herein, indicates that the therapeutically effective amount is administered periodically (as distinguished from a one-time dosc). In one embodiment, the PSMA targeted anticancer-phthalocyanine conjugate compound is administered periodically, e.g., at a regular interval (e.g., bimonthly, monthly, biweekly, weekly, twice weekly, daily, twice a day or three times or more often a day).

The administration interval for a single individual can be fixed, or can be varied over time, depending on the needs of the individual. For example, in times of physical illness or stress, or if disease symptoms worsen, the interval between doses can be decreased. Depending upon the half-life of the agent in the subject, the agent can be administered between, for example, once a day or once a week.

For example, the administration of the PSMA targeted anticancer-phthalocyanine conjugate compound described herein and/or the additional therapeutic agent can take place at least once on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least once on week 1, 2, 3, 4, 5, 6, 7, 8, 9, 10, 11, 12, 13, 14, 15, 16, 17, 18, 19 or 20, or any combination thereof, using single or divided doses of every 60, 48, 36, 24, 12, 8, 6, 4, or 2 hours, or any combination thereof. Administration can take place at any time of day, for example, in the morning, the afternoon or evening. For instance, the administration can take place in the morning, e.g., between 6:00 a.m. and 12:00 noon; in the afternoon, e.g., after noon and before 6:00 p.m.; or in the evening, e.g., between 6:01 p.m. and midnight.

A disclosed PSMA targeted anticancer-phthalocyanine conjugate compound and/or additional therapeutic agent can be administered in a dosage of, for example, 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day. Dosage forms (composition) suitable for internal administration generally contain from about 0.1 milligram to about 500 milligrams of active ingredient per unit. In these pharmaceutical compositions, the active ingredient will ordinarily be present in an amount of about 0.5-95% by weight based on the total weight of the composition.

The amount of disclosed PSMA targeted anticancer-phthalocyanine conjugate compound and/or additional therapeutic agent administered to the subject can depend on the characteristics of the subject, such as general health, age, sex, body weight and tolerance to drugs as well as the degree, severity and type of rejection. The skilled artisan will be able to determine appropriate dosages depending on these and other factors using standard clinical techniques.

In addition, in vitro or in vivo assays can be employed to identify desired dosage ranges. The dose to be employed can also depend on the route of administration, the seriousness of the disease, and the subject's circumstances. Effective doses may be extrapolated from dose-response curves derived from in vitro or animal model test systems. The amount of the compound can also depend on the disease state or condition being treated along with the clinical factors and the route of administration of the compound.

For treating humans or animals, the amount of disclosed PSMA targeted anticancer-phthalocyanine conjugate compound and/or additional therapeutic agent administered (in milligrams of compound per kilograms of subject body weight) is generally from about 0.1 mg/kg to about 100 mg/kg, typically from about 1 mg/kg to about 50 mg/kg, or more typically from about 1 mg/kg to about 25 mg/kg. In a preferred embodiment, the effective amount of agent or PSMA targeted anticancer-phthalocyanine conjugate compound is about 1-10 mg/kg. In another preferred embodiment, the effective amount of agent or PSMA targeted anticancer-phthalocyanine conjugate compound is about 1-5 mg/kg. The effective amount for a subject can be varied (e.g., increased or decreased) over time, depending on the needs of the subject. In other embodiments, the amount of disclosed PSMA targeted anticancer-phthalocyanine conjugate compound administered (in nmols of compound per kilogram of subject body weight) is generally from about 10 nmol/kg to about 1000 nmol/kg, typically from about 50 nmol/kg to about 500 nmol/kg, or more typically from about 100 nmol/kg to about 250 nmol/kg.

The PSMA targeted anticancer-phthalocyanine conjugate compound and/or additional therapeutic agent described herein can be administered to the subject in conjunction with an acceptable pharmaceutical carrier or diluent as part of a pharmaceutical composition for therapy. Formulation of the PSMA targeted anticancer-phthalocyanine conjugate compound to be administered will vary according to the route of administration selected. Suitable pharmaceutically acceptable carriers may contain inert ingredients that do not unduly inhibit the biological activity of the PSMA targeted anticancer-phthalocyanine conjugate compounds. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like.

The preparation of a pharmacological composition that contains active ingredients dissolved or dispersed therein is well understood in the art. Typically, such compositions are prepared as injectables as either liquid solutions or suspensions, however, solid forms suitable for solution, or suspensions, in liquid prior to use can also be prepared. Formulation will vary according to the route of administration selected (e.g., solution, emulsion, capsule).

The following example is for the purpose of illustration only and are not intended to limit the scope of the claims, which are appended hereto.

### EXAMPLE

In this Example, we developed a multifunctional theranostic approach that combines a cytotoxic drug (MMAE), a photosensitizer (Pc413) and a low molecular weight PSMA targeting ligand (PSMA-1-Cys-C6-Lys) into a single molecule, PSMA-1-MMAE-Pc413, that selectively and simultaneously delivers both chemotherapeutic drugs and photosensitizers to cancer cells. In addition, Pc413 emits near infrared light, therefore, this approach can also be used for near infrared fluorescence detection of cancers. Our results showed that PSMA-1-MMAE-PC413 can be selectively delivered to PSMA-expressing cancer cells. More importantly, the PSMA-targeted covalent combination of PDT and chemotherapy agents showed significantly improved antitumor activity compared to either PSMA-targeted MMAE treatment without PDT or PSMA-1-PC413 conjugate treatment with PDT.

### Methods

### In Vivo Antitumor Efficacy Studies

The effect of PSMA-1-MMAE-Pc413 was tested in mice bearing PC3pip tumors. Animals with tumor size at about 100 mm3 were used for the study (tumor volume=Length×width2/2). Animals were divided into 5 groups: (1) mice receiving PBS; (2) mice receiving PBS with PDT; (3) mice receiving 200 nmol/kg PSMA-1-MMAE-Pc413; (4) mice receiving 200 nmol/kg PSMA-1-MMAE-Pc413 with PDT; and (5) mice receiving 200 nmol/kg PSMA-1-Pc413 with PDT. Each group had 5 mice. Animals received drugs through tail vein injection on day 0 and day 7 and treated with 150 J/cm2 of 668 nm light at 24 h post-injection. Animals were imaged before and after PDT. Mice were monitored every other day for 90 days. Animals were euthanized at day 9. Data were reported as body weight over time and tumor size over time.

### Statistics

Student t-test was used to compare inter-group differences. A p value<0.05 was considered statistically significant for all comparisons.

### Results

### In Vivo Antitumor Activity Studies

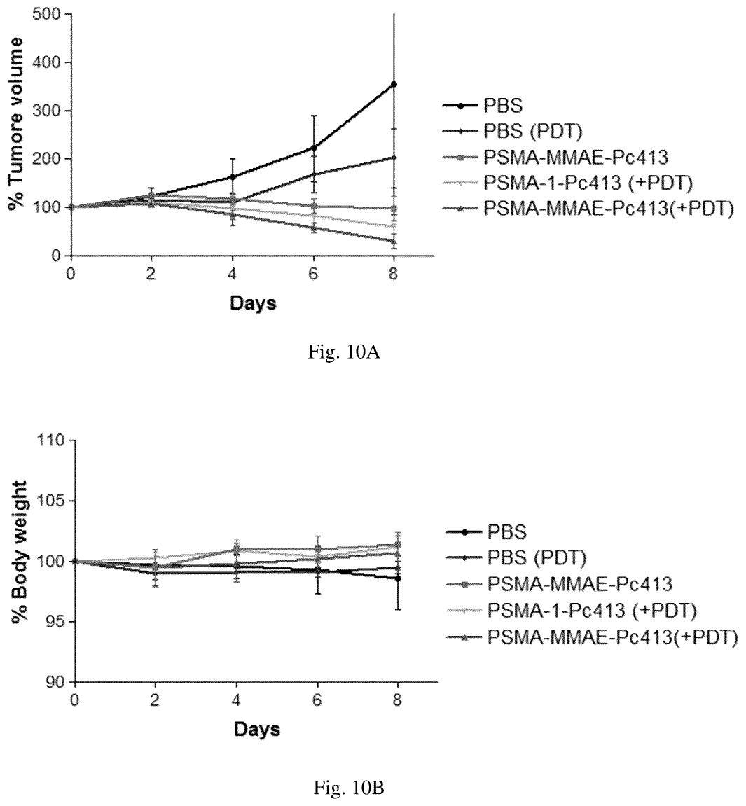

The effectiveness of PSMA-1-MMAE-Pc413 to eliminate prostate tumors was performed in mice bearing PC3pip tumors. Mice received 200 nmol of PSMA-1-MMAE-Pc413 through tail vein injection every 7 days. Mice were irradiated by 150 J/cm² of 668 nm light at the peak tumor accumulation of PSMA-1-MMAE-Pc413, which was 24 h post injection. Controls included i.v. administration of PBS with no light treatment, PBS with light treatment, 200 nmol/kg of PSMA-1-MMAE-Pc413 with no light treatment, and 200 nmol/kg of PSMA-1-Pc413 with 150 J/cm² of 668 nm. As shown in FIG. 10A, the greatest tumor growth inhibition was found in the animals receiving PSMA-1-MMAE-Pc413 with light irradiation (PSMA-1-MMAE-Pc413+PDT) as compared to the other 4 groups. Therefore, PSMA-1-MMAE-Pc413 with light improved the treatment outcome of PSMA-1-Pc413 with light irradiation and PSMA-1-MMAE-Pc413 with no light irradiation. In addition, the treatment did not cause any body weight loss (FIG. 10B).

Prostate cancer is highly heterogeneous, which will affect treatment response, drug resistance and clinical outcome. The use of combination therapies with different mechanisms of action will offer potential advantages over a single therapy and it can be an effective way to deal with the ‘heterogeneity’ of cancer cells. However, this is not a simple approach because different drugs may have different pharmacokinetics and do not necessarily get to the tumor at the same time and the drug can also go to other tissues in the body causing side effects. The use of anticancer drugs is therefore limited by unwanted side effects. To overcome these problems, we have developed a multifunctional molecule named PSMA-1-MMAE-Pc413 that combines chemotherapy, PDT, and imaging in a single molecule that is targeted to PSMA. PSMA is over expressed almost exclusively on prostate cancer. MMAE was selected as the chemotherapeutic drug because of its high potency, its wide use in antibody drug conjugates, its synergy with PDT and the fact that we have successfully targeted it to PSMA for the treatment of prostate cancer with a greater therapeutic index when compared to a PSMA-targeted antibody drug conjugate using MMAE.

No body weight loss was observed after the treatment (FIG. 10B). In in vivo antitumor activity studies, PSMA-1-MMAE-Pc413 demonstrated significantly greater efficacy for PC3pip tumors than single drug treatments (PSMA-1-MMAE-Pc413 without PDT or PSMA-1-Pc413 with PDT) (FIG. 10A). It is speculated that PSMA-1-MMAE-PC413+PDT may kill significantly more cancer cells than other treatment groups, leading to slower regrowth, i.e., less residual tumor cells, and/or therapeutic cures.

Our molecule combines both chemotherapy and PDT in a single targeted small molecule for a dual-therapeutic approach to combat prostate cancer. The selective targeting and rapid clearance of the molecule should dramatically reduce off-target toxicity while simultaneously increasing anti-cancer efficacy. For localized prostate cancer, minimally invasive fiber optics have been developed to irradiate the prostate gland with light, e.g., TOOKAD, providing the needed infrastructure for implementation of the PDT approach. In addition to localized NIR light irradiation, the efficacy of PSMA-1-MMAE-Pc413 can be extended to systemic cell killing by local release of active MMAE, which will overcome the problem that PDT cannot be used to treat large tumors due to limited light penetration. On the other hand, PDT will reduce the needed dose of the chemotherapeutic drug, therefore further reducing dose-related toxicity of MMAE. Compared to current clinical protocols for combination therapy of PDT and chemotherapy, our approach selectively delivers both drugs to cancer cells, reducing off-target toxicity related to untargeted drugs and achieving enhanced synergistic antitumor activity.

In conclusion, we have synthesized a multi-functional theranostic molecule for simultaneous and targeted delivery of both PDT and chemotherapy to prostate cancer cells. The multifunctional molecule showed selective uptake in PSMA-positive tumors and significantly enhanced (synergistic) antitumor activity was observed as compared to individual treatment with PDT or chemotherapy alone. It can be used in the operating room to help surgeons detect tumors using real-time FIGS and provide PDT and chemotherapy to kill any unresected cancer cells. It is also possible that the molecule can be used directly on prostate cancer patients that are not suitable for surgery, providing PDT and chemotherapy to cancer tissues.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, we claim:

1. A compound comprising the formula (1): 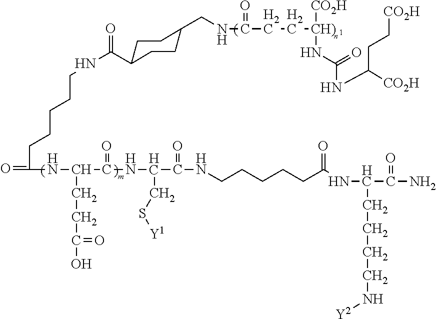
or a pharmaceutically acceptable salt thereof;
wherein m and n¹ are each independently 1, 2, 3, or 4;
Y¹ includes an anticancer agent that is directly or indirectly linked to the thiol group; and
Y² includes a phthalocyanine photosensitizer that is directly or indirectly linked to the amine group.

2. The compound of claim 1, wherein the phthalocyanine photosensitizer has the formula: 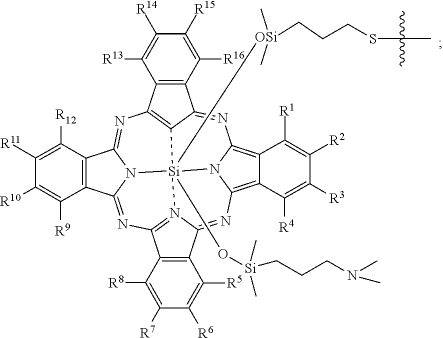
wherein R¹, R⁴, R⁵, R⁸, R⁹, R¹², R¹³, and R¹⁶ are each independently selected from the group consisting of hydrogen, halogen, nitro, cyano, hydroxyl, thiol, amino, and methyl; and
R², R³, R⁶, R⁷, R¹⁰, R¹¹, R¹⁴, and R¹⁵ are each independently selected from the group consisting of hydrogen, halogen, nitro, cyano, hydroxyl, thiol, amino, carboxy, aryl, heteroaryl, carbocyclyl, heterocyclyl, C₁₋₆ alkyl, C₁₋₆ alkenyl, C₁₋₆ alkynyl, C₁₋₆ alkoxy, C₁₋₆ acyl, C₁₋₆ alkylcarbonyloxy, C₁₋₆ carbocyclylalkyl, C₁₋₆ aminoalkyl, C₁₋₆ alkylamino, C₁₋₆ thioalkyl, C₁₋₆ alkylthio, C₁₋₆ hydroxyalkyl, C₁₋₆ alkyloxycarbonyl, C₁₋₆ alkylaminocarbonyl, and C₁₋₆ alkylcarbonylamino.

3. The compound of claim 1, wherein R¹, R², R³, R⁴, R⁵, R⁶, R⁷, R⁸, R⁹, R¹⁰, R¹¹, R¹², R¹³, R¹⁴, R¹⁵ and R¹⁶ are each independently selected from the group consisting of hydrogen, halogen, nitro, cyano, hydroxyl, thiol, amino, and methyl.

4. The compound of claim 1, wherein the phthalocyanine photosensitizer has the formula:

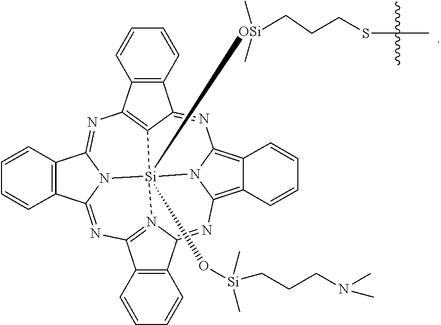

5. The compound of claim 1, wherein the phthalocyanine photosensitizer is linked to the amine group via a non-cleavable linker.

6. The compound of claim 5, wherein the linker includes a succinimidyl 4-(N-maleimidomethyl)cyclohexane-1-carboxylate (SMCC) linker.

7. The compound of claim 1, wherein the anticancer agent is linked to the thiol group via a protease cleavable or acid-labile linker.

8. The compound of claim 7, wherein the linker includes a maleimido-caproyl linker-valine-citrulline cleavable peptide-p-aminobenzyl carbamate spacer (MC-VC-PABC) protease cleavable linker.

9. The compound of claim 1, wherein the anticancer agent is monomethyl auristatin E (MMAE).

10. The compound of claim 1, having the formula: 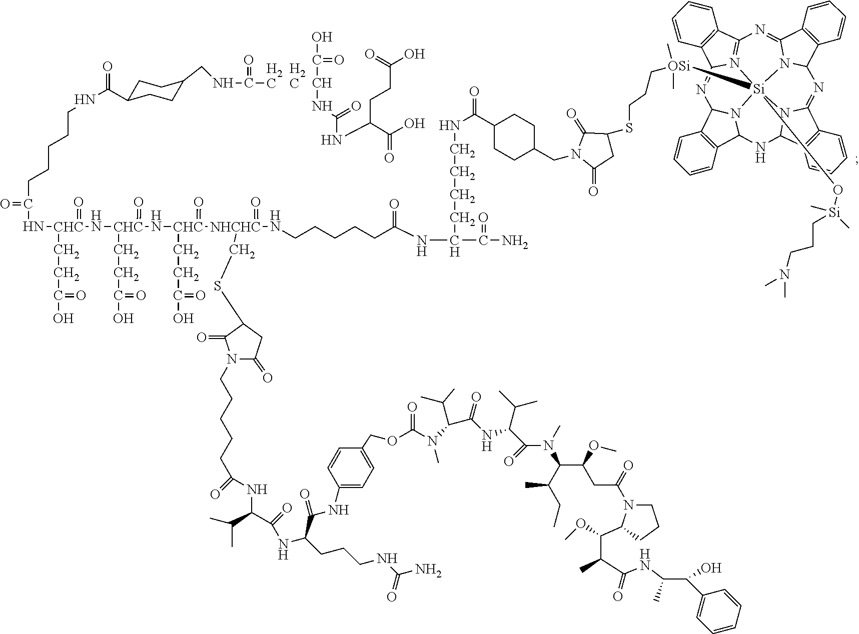
or a pharmaceutically acceptable salt thereof.

11. The compound of any of claim 1 having a selectivity for PSMA expressing cancer tissue versus non-PSMA expressing non-cancer tissue≥5 times or more.

12. A method of treating PSMA-expressing cancer in a subject in need thereof, the method comprising:
administering to the subject a therapeutically effective amount of a compound having general formula (I): 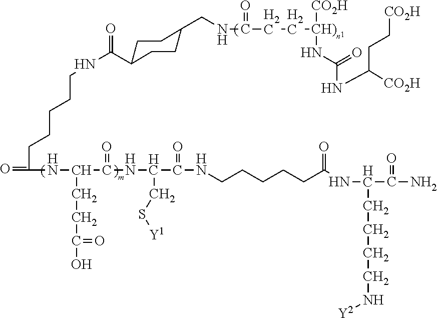
or a pharmaceutically acceptable salt thereof;
wherein m and n¹ are each independently 1, 2, 3, or 4;
Y¹ includes an anticancer agent that is directly or indirectly linked to the thiol group; and
Y² includes a phthalocyanine photosensitizer that is directly or indirectly linked to the amine group.

13. The method of claim 12, wherein the phthalocyanine photosensitizer has the formula:

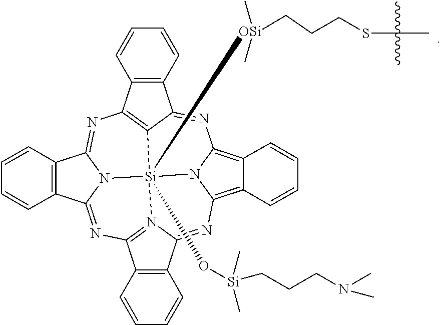

14. The method of claim 13, wherein the phthalocyanine photosensitizer is linked to the amine group via a non-cleavable linker.

15. The method of claim 13, wherein the anticancer agent is linked to the thiol group via a protease cleavable or acid-labile linker.

16. The method of claim 13, wherein the anticancer agent is monomethyl auristatin E (MMAE).

17. The method of claim 13, wherein the compound has the formula: 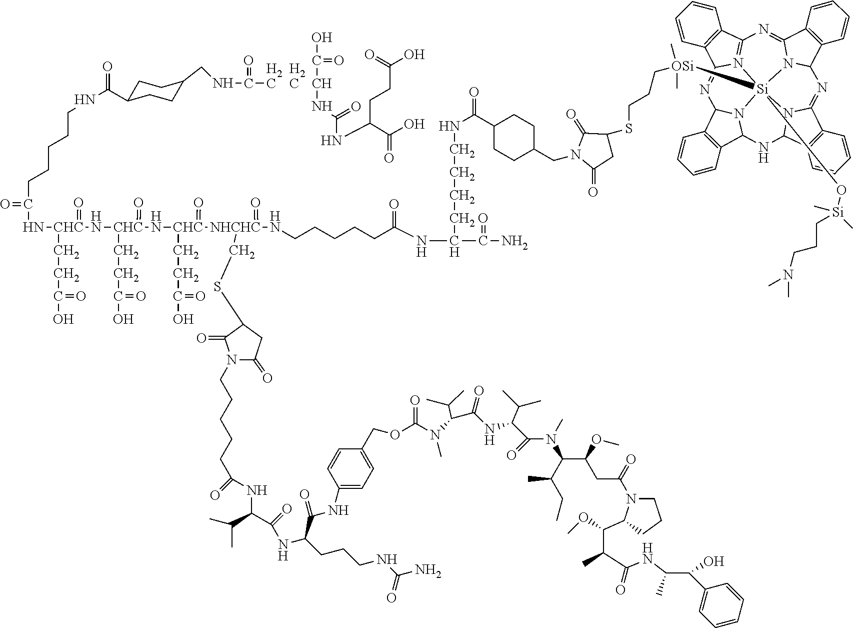
or a pharmaceutically acceptable salt thereof.

18. The method claim 12, further comprising detecting the compound bound to cancer cells in the subject.

19. The method claim 18, further comprising surgically resecting the detected cancer cells.

20. The method of claim 19, further comprising irradiating the site of surgical resection to ablate remaining or residual cancer cells.
